(19) United States
(12) Patent Application Publication
(10) Pub. No.: US 2024/0228938 A1
(43) Pub. Date: Jul. 11, 2024

(54) PROCESSING DEVICE FOR PROCESSING A LIQUID SAMPLE

(71) Applicant: CYTENA BIOPROCESS SOLUTIONS CO., LTD., Taipei City (TW)

(72) Inventors: Cheng-Han TSAI, Taipei City (TW); Da-Han KUAN, Taipei City (TW); Yeu-Farn SHIH, Taipei City (TW)

(21) Appl. No.: 18/559,658

(22) PCT Filed: May 10, 2022
(86) PCT No.: PCT/EP2022/062631

§ 371 Date: Nov. 8, 2023

(30) Foreign Application Priority Data
May 11, 2021 (LU) LU500144

(51) Int. Cl. C12M 1/00; C12M 1/32; C12M 1/34

(52) U.S. Cl. CPC C12M 41/14; C12M 23/12; C12M 23/38; C12M 41/40

(57) ABSTRACT

The invention relates to a processing device for processing a liquid sample, the processing device comprising at least one condenser element for condensing of vaporized liquid of the liquid sample and a housing that is positionable such that the condenser element is in fluid connection with a receptacle having the liquid sample, wherein the condenser element is part of the housing and/or arrangeable or arranged in a space of the housing, and at least one temperature changing device for creating a temperature difference between the condenser element and the vaporized liquid of the liquid sample.

The invention relates to a processing device for processing a liquid sample. Additionally, the invention relates to a processing system comprising such a processing device.

In a pharmaceutical process of biosimilar drugs, a cell line development process is required for most biopharmaceuticals. The cell line development is a method for selecting protein/antibodies, achieving a high yield of the protein/antibodies, and optimizing the quality thereof. However, the cell line development is on the other hand a very complicated, labor-intensive, and pricey process. Development time for most pharmaceutical factories is half a year.

A core tool required in the cell line development is a bioreactor for cell incubation. Starting from incubating single-cells in the process, the scale is continually expanded to a volume production scale through repeated transferring steps of selecting and scaling-up. Therefore, for different phases and different quantities of cells, bioreactors of different sizes ranging from a bioreactor at a microliter level to a large-scale bioreactor at a volume production level are required. However, there is a limitation on optimization of a cell incubating environment of a smaller-sized bioreactor. An ideal bioreactor generally needs to enable cells to continually grow in a suspension, dynamically monitor biosignals, and perform feedback control on dissolved oxygen content and a pH value. It is difficult to achieve the foregoing functions in a microliter-level bioreactor.

It is known to use a processing device and a multiwell carrier, which comprises a plurality of receptacles, e.g. 96 receptacles, for receiving liquid sample, in bioreactors. During the operation of the processing device is attached to the multiwell carrier. The processing device can be a device by means of which fluid can be supplied into or removed from the receptacle having the liquid sample in order to move and/or mix the liquid sample.

In processing the liquid sample in the bioreactor it is possible that a part of the liquid of the liquid sample vaporizes. Vaporization is even promoted when the liquid sample is moved and/or mixed as mentioned above. This leads to the problem that cell cultivation might be disturbed or even aborted due to a change of the physical condition of the liquid sample. In particular, the oxygen content and/or the pH-Value of the liquid sample change when the liquid vaporizes. The change of physical condition of the liquid is significant when the volume of the liquid sample is low, in particular in a microliter level. In those cases even a small vaporization of liquid sample leads to a significant change of physical condition.

The objective of the invention is to provide a processing device which reduces the risk of change of physical condition of the liquid, in particular during the operation of the processing device.

The objective is solved by a processing device for processing a liquid sample, the processing device comprising at least one condenser element for condensing of vaporized liquid of the liquid sample and a housing that is positionable such that the condenser element is in fluid connection with a receptacle having the liquid sample, wherein the condenser element is part of the housing and/or arrangeable or arranged in a space of the housing, and at least one temperature changing device for creating a temperature difference between the condenser element and the vaporized liquid of the liquid sample.

The inventive processing device has the advantage that it comprises a condenser element by means of which the vaporized liquid of the liquid sample condensates. Thereby, it is secured that the liquid reduction of the liquid sample by means of vaporizing is low and does not lead to a significant change in the physical condition of the liquid sample. In particular, a change of oxygen content and/or of the pH-Value can be reduced in comparison to known processing devices.

The liquid sample depends on the usage field of the processing device. If the processing device is used in a bioreactor, the liquid sample comprises a biological particle. The biological particle can be a cell or a microbe. The liquid sample can contain a liquid and at least one biological particle. The liquid can promote the growth of the biological particles, in particular cells or microbes, arranged in the liquid. If the processing device is used in a chemical reactor, the liquid sample can comprise one or more chemical reagents.

As mentioned above, processing of liquid sample can comprise the supply or removal of fluid, in particular gas, into the receptacle and/or mixing of the liquid sample in the receptacle and/or moving of the liquid sample and/or agitation of biological particles of the liquid sample. Moving of the liquid sample can be performed in order to apply shear stress to e.g. cells by means of the moved liquid. Mixing of the liquid sample is understood as a process in which the components of the liquid sample, in particular the biological particles, are moved relative to each other in such a way that a new arrangement pattern is created. The processing device can perform at least one of the aforementioned processing steps. The liquid processing can additionally or alternatively comprise a filtration of the liquid sample by means of a filter of the processing device.

A condenser element is an element by means of which the vaporized gas condenses. The liquid condenses on a surface of the condenser element. The condensed liquid can be attached to the condenser element at a first stage. In a second stage the condensed liquid can fall from the condenser element back into, in particular directly, the receptacle and, thus, onto the liquid sample. This can occur when the condensed liquid has reached a certain volume and/or weight so that the condensed liquid falls as a result on the gravity force acting on the condensed liquid.

The condenser element can be a part of the housing. As is explained below, this means that the condenser element and the housing are not separate components. Alternatively, the condenser element and the housing can be separate elements. In that case the condenser element can be or is arranged in the space of the housing. That means, the condenser element can be connected with the housing in a releasable manner.

The temperature changing device can be any device by means of which the temperature of the condenser element and/or the temperature of the vaporized liquid of the liquid sample can directly or indirectly be controlled. Said device is used to ensure that the temperature of the condenser element differs from the temperature of the vaporized liquid of the liquid sample. The temperature difference between the temperature of the condenser element and the temperature of the vaporized liquid of the liquid sample can be set to a predetermined value by means of the at least one temperature changing device. Alternatively, the temperature difference is not set to a predetermined value.

The processing device is configured such that the housing can be positioned such that it is in fluid connection with the receptacle. This means that the housing can be moved relative to the receptacle. In particular, the housing can be moved to a position in which the condenser element is in fluid connection with the liquid sample and thus also with the vaporized liquid of the liquid sample. A fluid connection between two components exists when the fluid can flow from one component into the other component or vice versa. The fluid connection permanently exists during the operation of the processing device That means, the fluid connection is not interrupted by means of components like valves.

According to an embodiment the condenser element is located such that the condensed liquid flows back into the receptacle and/or drops on the liquid sample being in the receptacle. Thus, a liquid reduction of the liquid sample can be prevented in an easy manner.

The processing device can comprise at least one control unit for controlling the at least one temperature changing device. The control unit can control the temperature changing device such that the condenser element and the vaporized liquid of the liquid sample of the liquid sample have different temperatures. In particular, the control unit can control the temperature changing device such that the temperature of the condenser element is lower than the temperature of the vaporized liquid of the liquid sample. For the case that the processing device or a processing system described later has several temperature changing devices all temperature changing devices can be controlled by the same control unit. Alternatively, the temperature changing devices can be controlled by different control units. The control unit is an electric or electronical control unit and/or can comprise at least one processor or is a processor.

The processing device can comprise a lid part. The condenser element can be part of the lid part. In said case the lid part is formed in one piece. A simple manner to create the condenser element is to form it by deep drawing the lid part. The condenser element can be formed such that it protrudes from the remaining lid part. In particular, the condenser element protrudes from the remaining lid part in a direction towards the receptacle. As a result, a compact processing device is achieved which consists of only several parts, in particular, only two parts, namely the lid part and a bottom part discussed below.

The condenser element can have a rectangular shape, in particular in its cross section. A part of the rectangular shape extends into the receptacle. Alternatively, the condenser element can have a tapered shape, in particular in its cross section. A part of the tapered shape extends into the receptacle. The provision of the tapered shape with its tip end arranged in the receptacle has the advantage that the condensed liquid moves to the tip end and falls from the tip end back into the receptacle. This has the advantage that at the tip end the volume and/or mass of the condensed liquid, which is needed that the condensed fluid falls down due to gravity force, is reached faster than in a rectangular shape so that the condensed liquid falls sooner back into the receptacle than in the embodiment in which the condenser element has the rectangular shape.

According to an embodiment the processing device can comprise a first temperature changing device for heating or cooling the lid part. The first temperature changing device enables that, in particular, for a predetermined time, the remaining lid part and the condenser element have different temperatures. In particular, the condenser element can have a lower temperature than the remaining lid part if the first temperature changing device heats the lid part, in particular the remaining part of the lid part not comprising the condenser element. Due to the temperature difference between the remaining lid part and the condenser element the condensation on the surface of the condenser element improves.

The first temperature changing device can be placed on the lid part. In particular, the first temperature changing device can be directly placed on the lid part, in particular the remaining part of the lid part not comprising the condenser element. The first temperature changing device can be formed such that it covers the lid part. This can be achieved when the first temperature changing device is formed as a heating or cooling plate. The condenser element extends from the remaining lid part in a direction away from the first temperature changing device. The temperature of the lid part can be controlled by the aforementioned control unit.

The first temperature changing device can comprise at least one through hole. The location of the through hole depends on the location of the condenser element. In particular the through hole can be located such that first temperature changing device does not cover the condenser element. The through hole can have the same dimension like the condenser element. Additionally or alternatively, the through-hole and the condenser element can be arranged coaxially to each other. This avoids that the condenser element is directly heated by the first temperature changing device but only the remaining lid part. The number of through holes can correspond with the number of condenser elements.

According to an embodiment the housing can have a bottom part. The bottom part can comprise at least one hollow extension tube that is at least partly arrangeable or arranged in the receptacle having the liquid sample. Additionally, the lid part and the bottom part can delimit the space. The hollow extension tube can also delimit a part of the space. In particular, the extension tube can delimit the part of the space in the circumferential direction of the extension tube. The extension tube can be open at one end, in particular the end facing the receptacle. This enables the liquid sample to be aspirated into or dispensed from the hollow extension tube into the receptacle, in particular into the liquid sample, by applying different pressure in the said space. In particular, this enables the liquid sample to be aspirated into or dispensed from the hollow extension tube into the receptacle, in particular into the liquid sample, by applying different pressure in the said space. The extension tube can protrude into the liquid sample when the processing device is attached on the receptacle.

The lid part and the bottom part can be firmly fixed together. In particular, the connection can be configured such that it cannot be solved without destroying the lid part and/or the bottom part. For example, the lid part and the bottom part can be welded. At the end of the assembling process, a compact processing device in modular form is achieved. That means, the processing device can consist of merely one component. This simplifies the usage of the processing device in a laboratory because the user has not to operate with several components.

The condenser element can correspond to at least a part of the bottom part. In particular, the condenser element can correspond to at least a part of the extension tube. In other words, the vaporized liquid of the liquid sample can condense on the extension tube if there is a temperature difference between the extension tube and the vaporized liquid of the liquid sample.

Additionally, or alternatively the aforementioned condenser element that is part of the lid part can be arranged in the extension tube or extends into the extension tube. The condenser element extends into the extension tube when there exists at least a plane that is arranged perpendicular to the length axis of the extension tube that comprises a part of the condenser element and a part of the extension tube. An embodiment comprising the aforementioned condenser elements has the advantage that in operation the vaporized liquid of the liquid sample condenses on the lid part, namely the condenser of the lid part, and the extension tube resulting in a low loss of liquid sample.

The thermal conductivity of the condenser element can differ from the thermal conductivity of the lid part and/or of the bottom part. In particular, the condenser element can have a thermal conductivity that is lower than the thermal conductivity of the lid part and/or of the bottom part. Thereby it is achieved that the condenser element has a lower temperature during the operation of the processing device than the lid part and/or the bottom part. This leads to that the temperature of the condenser element is lower than the temperature of the vaporized liquid of the liquid sample. Thus, condensing of vaporized liquid of the liquid sample by means of the condenser element improves.

The condenser element and the receptacle are formed such that a gap exists between the condenser element and the extension tube. This gap enables a fluid connection between the connection part and the receptacle. In other words, the gap is needed so that the pressure applied to the space, in particular to the space part surrounded by the extension tube, can be applied to the liquid sample.

The condenser element can have at least one step portion that is arranged outside the extension tube. In particular, the step portion can be arranged between the part of the condenser element that is arranged in the receptacle and the part of the connection element that is directly connected to the lid part. Outside of the extension tube means that the step portion is arranged such that there does not exist a plane perpendicular to the length axis of the extension tube comprising both the step portion and part of the extension tube. The step portion increases the surface area of the condenser element on which the vaporized liquid can condense. Thus, the step portion reduces the risk that the vaporized liquid is not brought back into the receptacle. The step portion can be arranged such that there exists a plane that comprises a part of the step portion and a part of the space delimited by the extension tube. In such an embodiment the condensed liquid falls from the step portion by means of gravity directly into the receptacle.

The processing device comprises a connection part for fluidically connecting the space to a pressure control system. The pressure control system is configured to provide a positive or negative pressure to the processing device, in particular, to the space. Thus, the connection part ensures that the provided positive or negative pressure is applied to the space of the processing device and, thus, via the extension tube on the liquid sample. Additionally, the pressure control system can be configured that the positive or negative pressure is applied alternately to the processing device. The negative or positive pressure can be applied multiple times to the processing device. By applying the processing device with positive or negative pressure in the aforementioned manner, it is secured that the liquid sample located in the receptacle is processed, in particular mixed.

The pressure control system can comprise at least one gas tank. In particular, the pressure control system can comprise a first gas tank having positive pressure and a second gas tank having negative pressure. A positive pressure is a pressure that is above the atmospheric pressure. A negative pressure is a pressure that is below the atmospheric pressure. Additionally or alternatively the pressure control system can comprises one or more valves for controlling the fluid flow to the processing device and/or one or more pumps for creating the positive or negative pressure.

The processing device can comprise a further connection part for, in particular fluidically, connecting the space with a measurement device. The measurement device can be a pressure sensor for measuring the pressure within the space. The measurement result can be used for controlling the pressure controlling system discussed above.

According to an embodiment the lid part can comprise a circumferential rim that surrounds a part of the bottom part. In particular, the rim surrounds the bottom part in a circumferential direction of the bottom part. A bottom plate of the bottom part can be directly connected to the circumferential rim. The extension tube extends from the bottom plate in a direction towards the receptacle. The extension tube can extend further than the circumferential rim along the length axis of the extension tube. This enables that the extension tube extends into the receptacle as it is discussed below more in detail.

The connection part and/or the further connection part can protrude from the lid part. Additionally, the connection part and/or the further connection part can protrude from the same lid part side. Both connection parts can protrude in the same direction from the lid part side.

According to an embodiment the processing device can comprise a bottom part that comprises several extension tubes. Additionally, the processing device can comprise several condenser elements. In particular, the number of extension tubes can correspond to the number of condenser elements. Thus, at least one extension tube, in particular merely one extension tube, extends into one receptacle. The provision of several condenser elements ensures that at least a part of the vaporized liquid of the liquid sample being arranged in each receptacle falls back into the respective receptacle.

According to an embodiment the processing device can comprise a lid part that comprises a first wall portion and a second wall portion. The first wall portion delimits the first chamber. The second wall portion and the bottom part delimit the second chamber that is fluidically connected to the first chamber. In this embodiment the space discussed above comprises the first chamber and the second chamber.

It has been observed that receptacles arranged next to the rim of the processing device have a higher evaporation rate than receptacles that are arranged further away from the rim of the processing device. This results as the receptacles arranged next to the rim of the processing device have a higher temperature than the receptacles arranged further inside the multiwell carrier because the rim is in contact with the outside environment. The receptacles arranged next to the rim of the processing device are also in more direct contact with ambient air, meaning it is more prone to losing vapor. Furthermore, the temperature adjustment device underneath the receptacle is also not perfect in terms of heat distribution. By having the connection part on the same side of the lid part as the rim, the differing condition between the rim of the processing device and the center portion of the processing device is reinforced and worsened. A receptacle is considered as “next to the rim” when no other receptacle is arranged between the rim and the said receptacle along the horizontal plane of the multiwell carrier. The receptacles that are next to the rim are receptacles arranged at the outer margin of a matrix form for a multiwell carrier where several receptacles are arranged in a matrix form.

A provision of a gas inlet, in particular air inlet, adjacent to a receptacle arranged next to the rim can worsen the situation as the stream of the entering gas transfer the vaporized gas from one receptacle to another receptacle, resulting in undesired contamination. The provision of the first wall portion with the first chamber achieves an improved gas flow that does not reinforce and in fact counters the aforementioned disadvantages. In particular, it is possible to achieve similar temperatures for the receptacles next to the rim in comparison to the receptacles arranged inside the multiwell carrier. Thus, the evaporation rates for the receptacles near the rim can be reduced. Additionally, this provision avoids transporting the evaporated liquid of the receptacles next to the rim to neighboring receptacles as the gas flow is directed outward so that gas passing through the receptacle next to the rim does not pass through another receptacle afterwards.

The lid part can have at least one hole for fluidically connecting the first chamber and the second chamber. The one or more holes can be arranged in the second wall portion. A plane can exist that comprises at least one hole or several holes wherein the plane is perpendicular to the length axis of the extension tube. The plane can also comprise a part of the second wall portion. By arranging holes as such, it is ensured that gas, in particular air, entering or leaving the second chamber flows in a direction parallel to the length axis of the extension tube.

The first wall portion can comprise an attachment portion for attaching the first wall portion on the second wall portion, wherein a section of the second wall portion that is delimited by the attachment portion comprises at least one hole. The attachment portion can protrude from the remaining first wall portion towards the second wall portion. Thus, in an attached state, a gap exists between the remaining first wall portion and the remaining second wall portion. The section of the second wall portion also delimits the first chamber. The remaining part of the second wall portion does not delimit the first chamber

The attachment portion surrounds a part of the first chamber. The first wall portion and the second wall portion can be, in particular firmly, connected to each other. In particular, the attachment portion of the first wall portion can be welded to the second wall portion.

One or more holes can be arranged in the central region of the lid part. The central region of the lid part is a region that does not comprise the circumferential rim of the lid part. Additionally, said region is arranged distant from the rim of the lid part and comprises a center of the lid part. The rim has several portions, in particular four rim portions, each of which extends along a side of the lid part. The one or more holes can be arranged between the opposite rim portions of the lid part. The rim portions can be opposite to each other with respect to a plane that extends parallel to the two rim portions. The at least one hole can be arranged in a section of the lid part, which is arranged between two extension tubes. At least one of the extension tubes, in particular both extension tubes, is not arranged next to a rim portion of the lid part. Thus, it is ensured that the extension tubes are arranged in a central region of the lid part. Said hole positioning ensures that the at least one hole is arranged distant from the rim of the lid part so that gas exchange does not occur in a region that is near the rim of the lid part. This avoids the aforementioned disadvantages.

The first wall portion can be arranged such that gas flows from the first chamber to the second chamber when a positive pressure is applied by the pressure control system. The positive pressure acts in the extension tube. Alternatively or additionally, the first wall portion can be arranged such that gas flows from the second chamber to the first chamber when a negative pressure is applied by the pressure control system. The negative pressure acts in the extension tube. Thus, the first chamber is arranged between the pressure control system and the second chamber in terms of gas flow.

According to an embodiment the first wall portion can be, in particular firmly, connected to the second wall portion. The first wall portion and the bottom part can be arranged opposite to each other with respect to the second wall portion. The first wall portion can be arranged on the second wall portion. The second wall portion can be, in particular firmly, connected to the bottom part and/or the condenser element can be part of the second wall portion. Firmly connected means that the two components are connected such that they move together when one of the components is moved. Additionally, the components cannot move relative to each other. The processing device has a stacked configuration comprising of the lid part, in particular the first wall portion and the second wall portion, and the bottom part.

The connection part can extend from the first wall portion. Thus, it is ensured that the pressure applied by the pressure control system first acts on the first chamber and afterwards on the second chamber.

According to another aspect of the invention a processing system is provided. The processing system comprises a receptacle and an inventive processing device. In the processing system, the housing is positioned such that the condenser element is in fluid connection with the receptacle. The processing device can be arranged on the receptacle or multiwell carrier comprising several receptacles. In said position at least a part of the extension tube can be arranged in the receptacle. The extension tube can extend into the receptacle so that the space of the processing device is in fluid connection with the liquid sample arranged in the receptacle. As mentioned before, the extension tube can protrude into the liquid sample. Thus, the extension tube can have a hollow portion in which the liquid sample is arranged. Additionally, the extension tube can have another hollow portion, in particular arranged above the hollow portion, in which no liquid sample is arranged but the vaporized liquid of the liquid sample.

The processing device is arranged above, in particular on top of, the receptacle. In particular, the processing device is arranged on the receptacle such that the circumferential rim is in contact with the receptacle. The circumferential rim contacts an upper part of the receptacle.

The receptacle can be part of a multiwell carrier. The multiwell carrier comprises a plurality of receptacles. The processing device that comprises the bottom part with several extension tubes can be placed on the multiwell carrier. Thus, at least one extension tube at least partly extends into one receptacle. In that embodiment the circumferential rim can come in contact with an upper part of the multiwell carrier. The plurality of receptacles can be arranged in a matrix form.

According to an embodiment, the processing system can comprise a second temperature changing device for heating or cooling the receptacle. Thus, the second temperature changing device heats or cools the liquid sample and/or the vaporized liquid of the liquid sample. Thus, by means of the second temperature device, it can be ensured that the temperature of the vaporized liquid of the liquid sample has a different temperature than the condenser element. The first temperature changing device and the second temperature changing device can be arranged opposite each other with respect to the receptacle and/or the bottom part. This enables a compact structured processing system.

The receptacle can be, in particular directly, arranged on the second temperature changing device. The receptacle can be formed so that it covers the second temperature changing device. The second temperature changing device can be a heating plate. The temperature of the heating plate can be controlled by a control unit.

The processing system can also have a control unit for controlling the pressure control system. In particular, the control unit can control whether a positive or negative pressure is applied by the pressure control system on the processing device. Said control unit can also control the first temperature changing device and/or the second temperature changing device.

The at least one control unit can control the first temperature changing device such that the temperature of the lid part and/or bottom part increases or decreases, in particular by 30 to 60 degree Celsius. The temperature of the bottom part can be changed by the first temperature changing device because the lid part is connected with the bottom part as mentioned above. The connection can be made such that thermal energy can be conducted between the lid part and the bottom part. If the control unit increases the temperature of the lid part, the temperature of the remaining part of the lid part and the bottom part is higher than the temperature of the condenser element. The vaporized liquid of the liquid sample is heated by at least the bottom part, in particular the extension tube, so that the temperature difference between the vaporized liquid of the liquid sample and the condenser element increases. The temperature of the condenser element corresponds to the ambient temperature, in particular at least at the begin of operation when the heating device and/or the further heating device start to heat the lid part and/or the receptacle. The condenser element surface on which the vaporized liquid is condensed is arranged in the surface surrounded by the extension tube when the condenser element is part of the lid part.

The at least one control unit can control the second temperature changing device such that the temperature of vaporized liquid of the liquid sample increases, in particular by 30 to 60 degree Celsius. The condenser element can remain at e.g. the ambient temperature so that the temperature difference between the condenser element and the vaporized liquid sample increases.

The at least one control unit can control the first temperature changing device such that the temperature of the lid part increases and controls the second temperature changing device such that the temperature of the vaporized liquid of the liquid sample increases. Alternatively or additionally, the at least one control unit can control the first temperature changing device such that the temperature of the lid part decreases and controls the second temperature changing device such that the temperature of the vaporized liquid of the liquid sample increases. In both cases the temperature difference between the condenser element and the vaporized liquid sample of the liquid sample can be easily increased.

The processing system can have temperature sensors in order to measure the temperature of the lid part and/or the bottom part and/or receptacle and/or the liquid sample.

In the figures, the subject-matter of the invention is schematically shown, wherein identical or similarly acting elements are usually provided with the same reference signs.

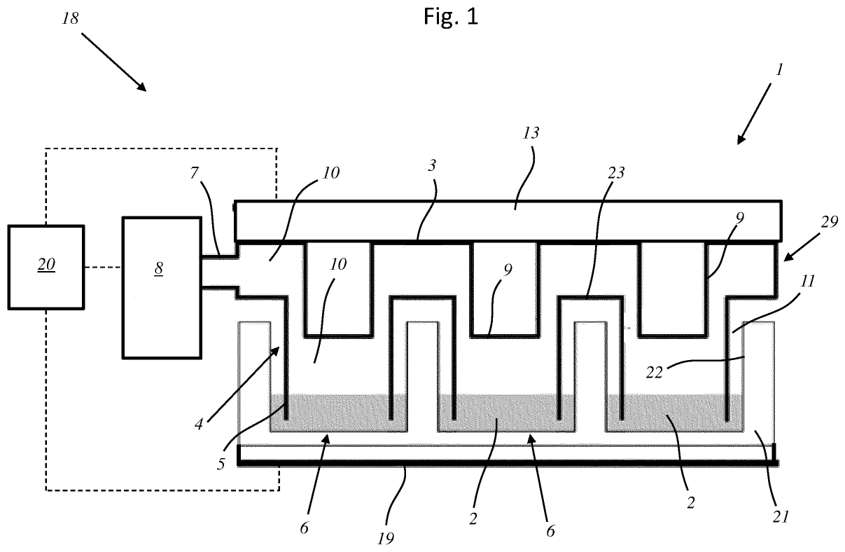

FIG. 1 shows a side view of the processing system with a processing device according to a first embodiment,

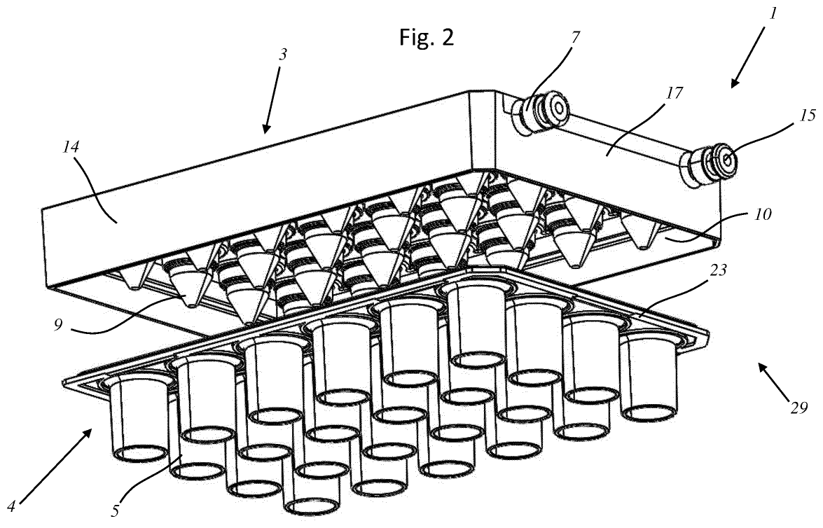

FIG. 2 shows an explosion view of a processing device according to a second embodiment,

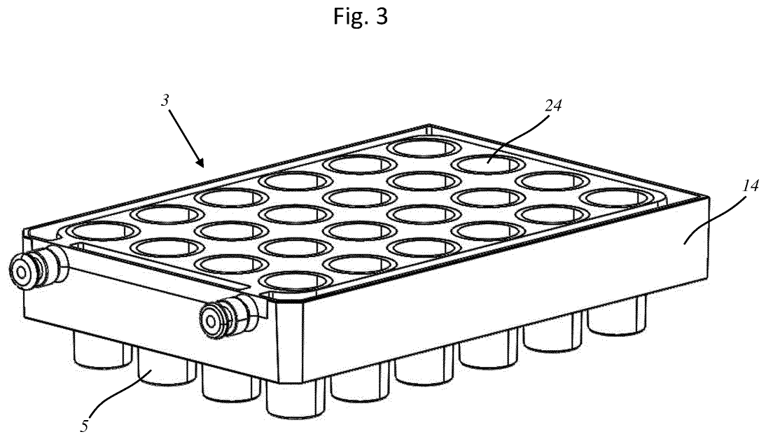

FIG. 3 shows a perspective view of the processing device according to the second embodiment from above,

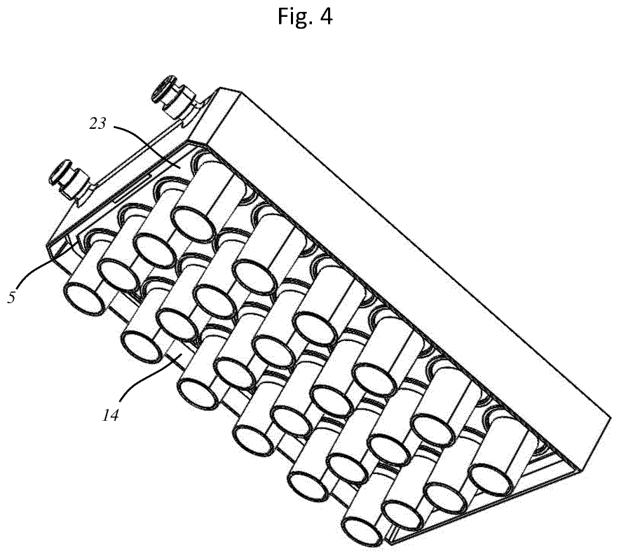

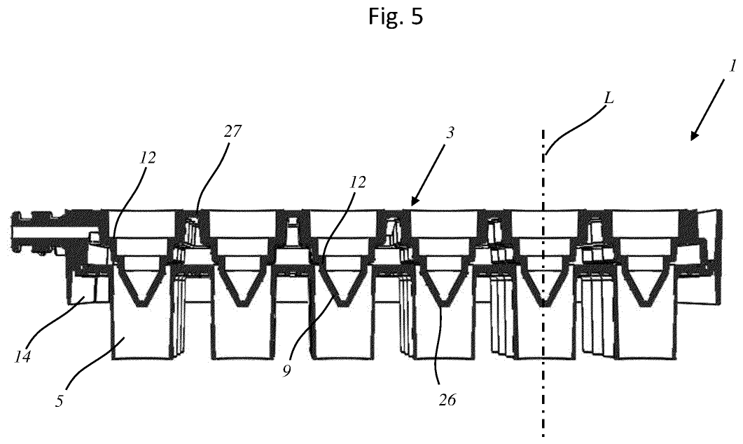

FIG. 4 shows a perspective view of the processing device according to the second embodiment from below,

FIG. 5 shows a side view of a cross section of the processing device according to the second embodiment,

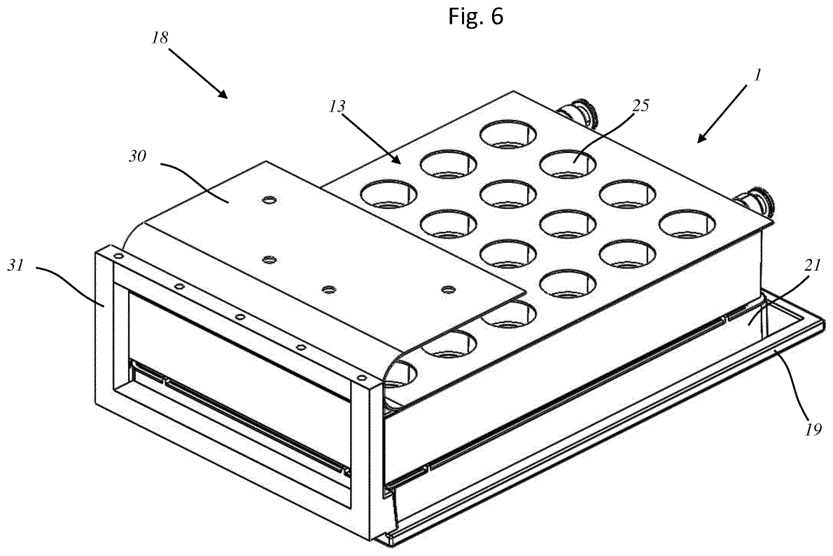

FIG. 6 shows a perspective view of a processing system with the processing device,

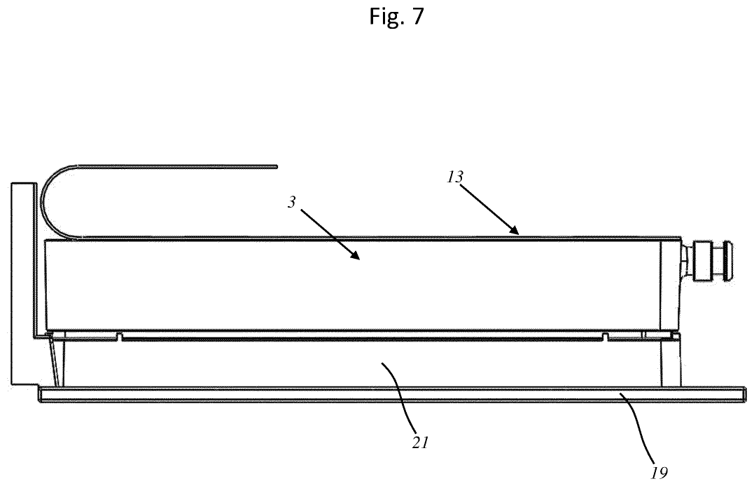

FIG. 7 shows a side view of the processing system shown in FIG. 6,

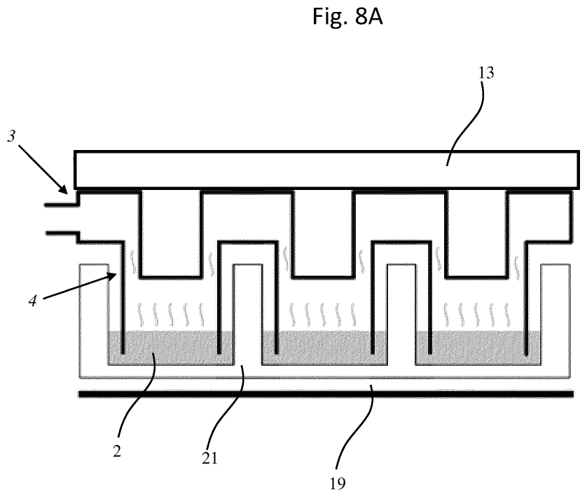

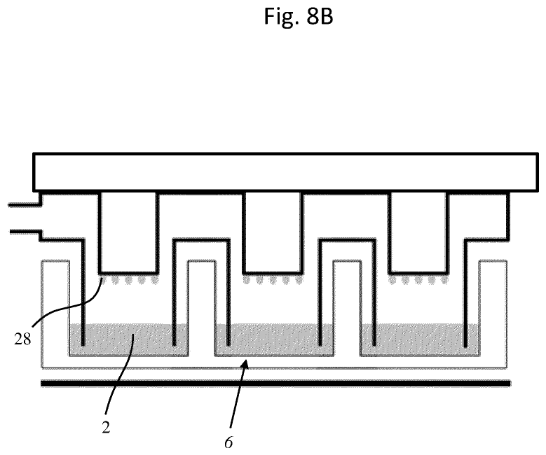

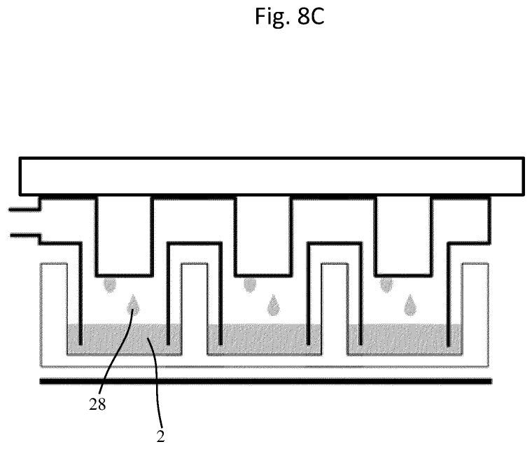

FIG. 8A shows the first stage of the condensing process by using the processing system as shown in FIG. 1.

FIG. 8B shows the second stage of the condensing process by using the processing system as shown in FIG. 1,

FIG. 8C shows the third stage of the condensing process by using the processing system as shown in FIG. 1,

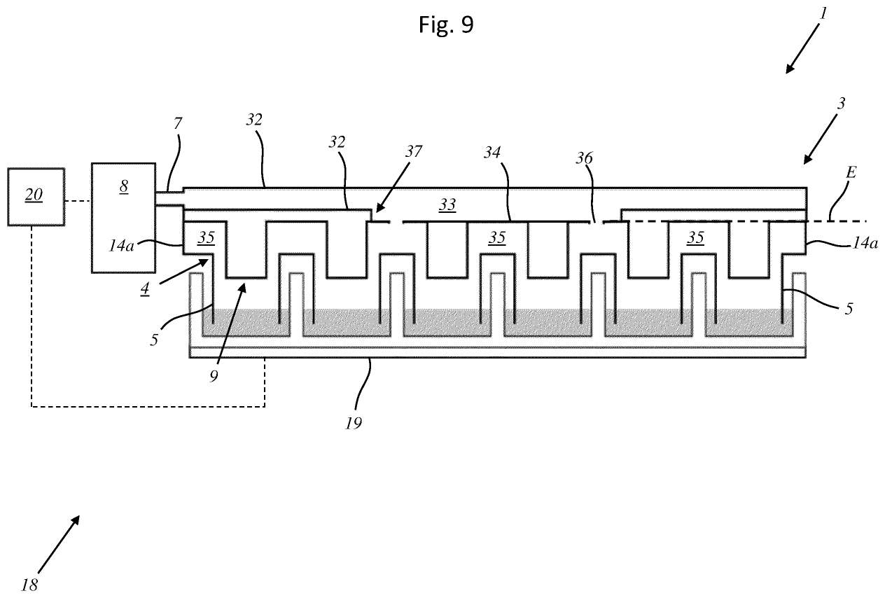

FIG. 9 shows a processing system with a processing device according to a third embodiment,

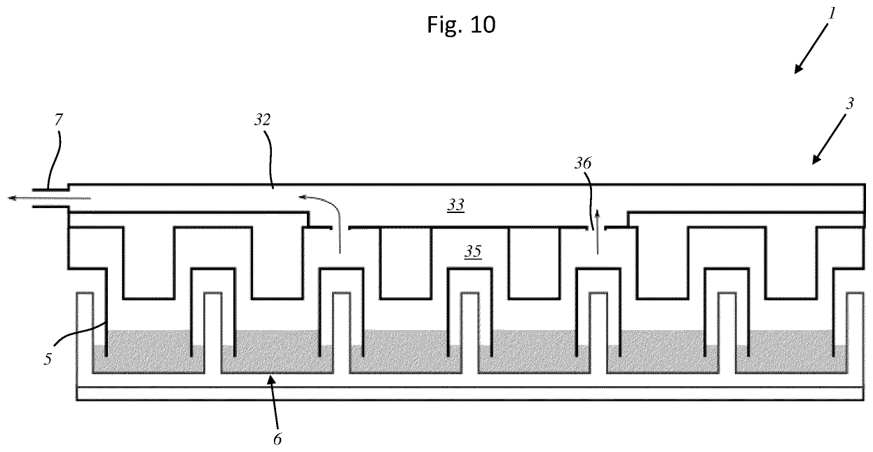

FIG. 10 shows the processing system of FIG. 9 in which a negative pressure is applied in the extension tube.

FIG. 1 shows a side view of a processing system 18 with a processing device 1 according to a first embodiment and a multiwell carrier 21. The multiwell carrier 21 comprises a plurality of receptacles 6. Each of the receptacles 6 comprises a liquid sample 2.

The processing device 1 is used for processing a liquid sample 2 and comprises a housing 29. The housing 29 comprises a lid part 3 and a bottom part 4. The bottom part 4 comprises several hollow extension tubes 5 that are partly arranged in respective receptacles 6, each of which has a liquid sample 2. The extension tubes 5 extend from a bottom plate 23 towards the receptacle 21. The processing device 1 comprises several condenser elements 9 for condensing vaporized liquid of the liquid sample 2, respectively. The condenser element 9 is part of the lid part 3 and is partly arranged in the hollow extension tube 5. All condenser elements 9 and extension tubes 5 are identically formed so that in the following the structure and function of only one condenser element and extension tube 5 are described. Additionally, the processing device 1 comprises a first temperature changing device 13 for creating a temperature difference between the temperature of the condenser element 9 and the temperature of a vaporized liquid of the liquid sample 2.

The processing device 1 comprises a lid part 3 that is fixedly connected with the bottom part 4. The lid part 3 and the bottom part 4 delimit a space 10 therebetween. Additionally, the hollow extension tubes 4 delimit the space 10 in circumferential direction of the respective extension tube 5. The extension tube 5 extends in the receptacle 6 such that it protrudes into the liquid sample 2 and/or has a cylindrical shape. The cross section of the extension tube 5, in particular the outer diameter of the extension tube, is smaller than the cross section of a receptacle wall 22, in particular the inner diameter of the receptacle wall 22. Thus, a gap 11 exists between the extension tube 5 and the receptacle wall 22. The extension tube 5 has an opening at its end opposite to the bottom plate 23. The space 10 is fluidically connected with the receptacle by means of the said opening.

Additionally, the processing device 1 comprises a connection part 7 that fluidically connects the space 10 with a pressure control system 8 of the processing system 18. The pressure control system 8 is configured to apply a positive or negative pressure. Due to the fluid connection between the space 10 and the pressure control system 8 by means of the connection part 7, the pressure control system 8 applies a positive or negative pressure on the space 10. By applying a negative pressure in the space 10 the liquid sample 2 is aspirated into the extension tube 5 and by applying a positive pressure in the space 10 the liquid sample 2 arranged in the extension tube 5 is dispensed into the receptacle 6. The space 10 is designed such that by applying a pressure in the space 10, said pressure is applied to all receptacles 6 by means of the respective extension tube 5.

The processing device 1 is placed on the multiwell carrier 21 as will be explained later in more detail. As mentioned before, the multiwell carrier 21 comprises a plurality of receptacles 6 wherein the number of receptacles 6 corresponds to the number of extension tubes 5.

The condenser element 9 is part of the lid part 3. The condenser element 9 has a rectangular shape wherein only a part of the condenser element 9 is arranged in the receptacle 6.

The first temperature changing device 13 is, in particular directly, arranged on the lid part 3. As the condenser element 9 extends from the remaining lid part 3 towards the multiwell carrier 21, at least the part of the condenser element 9 arranged in the receptacle 6 has a different temperature than the remaining lid part 3 when the first temperature changing device 13 heats the lid part 3. The lid part 3 heats gas arranged in the space 10. The first temperature changing device 13 can be a heating plate that covers the lid part 3. As the bottom part 4 is connected to the lid part 3, the bottom part is also heated when the lid part 3 is heated by the temperature changing device 13. In said case the bottom part 4, in particular the extension tube 5, also heats the vaporized liquid of the liquid sample 2 and thus increases the temperature difference between the condenser element 9 and the vaporized liquid of the liquid sample.

The processing system 18 comprises a second temperature device 19. The second temperature device 19 is used to heat the multiwell carrier 21, in particular the liquid samples 2 arranged in the receptacles 6. The multiwell carrier 21 is arranged on the second temperature device 19. In particular, the multiwell carrier 21 is placed on the second temperature device 19. The second temperature device 19 can be a heating plate. The first temperature device 13 and the second temperature device 19 can be arranged opposite to each other with regard to the multiwell carrier 21 and/or the bottom part 4. The second temperature changing device 19 can heat the multiwell carrier 21 and, thus, the liquid sample 2 and the vaporized liquid sample arranged in the multiwell carrier 21.

The processing system 18 comprises a control unit 20 for controlling the first temperature changing device 13 and the second temperature changing device 19. Alternatively, the first temperature changing device 13 and the second temperature changing device 19 can be controlled by different control units 20 that are not shown. The control unit 20 can control the first temperature changing device 13 and the second temperature changing device 19 such that the lid part 3 and/or the multiwell carrier 21 reach a predetermined temperature. The processing system 18 comprises temperature sensors (not shown) for measuring the temperature of the lid part 3 and/or bottom part 4 and/or the multiwell carrier 21 and/or the liquid sample 2. The control unit 20 controls the first and/or second temperature changing devices 13, 19 based on the measured temperature values.

Additionally, the control unit 20 can control the pressure control system 8. Alternatively, the pressure control system 8 can be controlled by a different control unit. The control unit is electrically or electronically connected to the heating device 13 and the further heating device 19. Additionally, the control unit is electrically or electronically connected to the pressure control system. The electrical connections are shown in dotted lines in FIG. 1.

FIG. 2 shows an explosion view of a processing device 1 according to a second embodiment. This processing device 1 differs from the processing device 1 shown in FIG. 1 in the shape of the condenser elements 9. In the embodiment shown in in FIG. 2 the condenser elements 9 have a tapered shape, respectively.

The processing device 1 comprises a further connection part 15. The further connection part 15 is used to fluidically connect the space 10 with a measurement device (not shown). The measurement device can be a pressure sensor. The pressure sensor measures the pressure within the space 10. Additionally or alternatively, the measurement device can be a temperature sensor for measuring the temperature of the lid part 3. The connection part 7 and the further connection part 15 extend from the same lid part side 17 in the same direction.

The lid part 3 has a circumferential rim 14. The circumferential rim 14 delimits the space 10 in circumferential direction of the processing device 10. The circumferential rim 14 extends in the same direction as the condenser element 9.

The condenser elements 9 are arranged in a matrix structure. That means, there exists several rows and columns each having several condenser elements 9. The condenser elements 9 are arranged in distance adjacent to each other. The number and arrangement of the extension tubes 5 are identical to the number and arrangement of the condenser elements 9.

FIG. 3 shows a perspective view of the processing device according to the second embodiment from above. Said FIG. 3 shows that the condenser elements 9 shown in FIG. 2 can be formed by deep drawing the lid part 3. Thus, there exist recesses 24 in the locations where the condenser elements 9 are arranged.

FIG. 4 shows a perspective view of the processing device 1 according to the second embodiment from below. From said view it is evident that the bottom plate 23 is firmly connected to the rim 14. Additionally, it is evident that the extension tube 5 extends further than the rim 14 in the direction away from the lid part 3.

FIG. 5 shows a side view of a cross section of the processing device 1 according to the second embodiment. A part of the tapered condenser element 9 is arranged in the extension tube 5. The condenser element 9 comprises two step portions 12. The step portions 12 are arranged outside of the extension tube 5. In particular, both step portions 12 are arranged at a location of the condenser element 9 that is between the tip 26 of the tapered condenser element 9 and the connection portion 27 in which the condenser element 9 is connected to the lid part 3. The step portions 12 are displaced with regard to each other along a length axis L of the extension tube.

FIG. 6 shows a perspective view of a processing system 18 with the processing device 1 and FIG. 7 shows a side view of the processing system shown in FIG. 6. The processing device 1 can be configured according to the first embodiment or the second embodiment described above or according to the third embodiment described below. That means, the condenser element 9 can have a rectangular shape or a tapered shape.

The first temperature changing device 13 is formed as a heating plate and attached to the lid part 3. The first temperature changing device 13 has through holes 25 in the area where the condenser element 9 is arranged. Thus, the heating plate does not cover the condenser element 9. The first temperature changing device 13 comprises a connection portion 30 by means of which it can be connected to other components (not shown) of the processing system 18. The processing device 1 is placed by means of the rim 14 on the multiwell carrier 21. The multiwell carrier 21 is arranged on the second temperature changing device 19. The second temperature changing device 19 has an extension 31 that extends in the vertical direction. The extension 31 simplifies the movement of the processing system 18.

FIGS. 8A to 8C show different stages of a condensing process by using the processing system 1 as shown in FIG. 1. The condensing process is basically the same for the processing device according to the second embodiment shown in FIGS. 2 to 8 if used instead of the processing system as shown in FIG. 1.

FIG. 8A shows the first stage of the condensing process in which the liquid of the liquid sample 2 starts to vaporize. The begin and extend of vaporization of liquid of liquid sample 2 can be influenced by controlling the first temperature changing device 13 and the second temperature device 19. In particular, the first temperature changing device 13 and the second temperature device 19 can be controlled such to have a predetermined temperature. In the present case both temperature changing devices 13, 19 heat the respective component.

The bottom part 4 is heated by the lid part 3 and has, in particular basically, the same temperature as the lid part 3. The extension tubes 4 and/or the remaining lid part heat the vaporized liquid sample. The second temperature changing device 19 heats the receptacle 6 and, thus, the liquid sample 2 and the vaporized liquid of the liquid sample.

As the condenser element 9 extends from the lid part 3 towards the multiwell carrier 21, the condenser element 9 has a different temperature than the remaining lid part 3 in direct contact with the heating device 13. In particular, the condenser element 9 has a lower temperature than the remaining lid part 3 and/or multiwell carrier 21 and/or the liquid sample 2 and/or the vaporized liquid sample. At the beginning of the operation the temperature of the condenser element 9 is ambient temperature.

FIG. 8B shows the second stage of the condensing process by using the processing system 1 as shown in FIG. 1. In the second stage, condensed liquid 28 is arranged on the condenser element 9. In particular, the condensed liquid 28 is arranged on the part of the condensed element 9 that is arranged in the receptacle 6.

FIG. 8C shows the third stage of the condensing process by using the processing system 1 as shown in FIG. 1. At the third stage the condensed liquid 28 has such a volume and/or mass that it falls down due to gravity force back into the receptacle 6, and thus onto the liquid sample 2.

FIG. 9 shows a processing system 18 with a processing device 1 according to a third embodiment. The processing device 1 differs from the processing device shown in FIG. 1 in the structure of the lid part 3. Another difference is that no temperature changing device is attached to the lid part 3. However, in an embodiment (not illustrated), the temperature changing device can be arranged on the lid part 3 as shown in the other embodiments.

The lid part 3 comprises a first wall portion 32 and a second wall portion 34 that protrudes from the remaining first wall portion 32 towards the second wall portion 34. The first wall portion 32 delimits the first chamber 33 and comprises the connection part 7 which is fluidically connected to the pressure control system 8. The second wall portion 34 and the bottom part 4 delimit a second chamber 35. The first wall portion 32 and the second wall portion 34 are firmly connected to each other. Additionally, the first wall portion 32 is arranged on the second wall portion 34.

The first wall portion 32 comprises an attachment portion 36. The attachment portion 36 is arranged on the second wall portion 34 and in contact with the second wall portion 34. The holes 36 for ensuring fluid connection between the first chamber 33 and the second chamber 35 are arranged in a particular section of the second wall portion 34. This section of the second wall portion 34 is in contact with the attachment portion 36. The holes 36 are arranged in a plane E that is perpendicular to the length axis L of the extension tube 5. The hole 36 is arranged in a region of the second wall portion 34 that connects the condenser elements 9 and/or is arranged between the condenser elements 9.

The lid part 3 has a rim 14 that comprises several rim portions. The rim portions extend along a side of the lid part 3, respectively. As it is evident from FIG. 9, the holes 36 are arranged in a region of the lid part 3 that is arranged between two rim portions 14a and/or between two extension tubes 5 each of which is arranged next to the respective rim portion 14a.

FIG. 10 shows the processing system of FIG. 9 in which a negative pressure is applied in the extension tubes 5 by the pressure control system 8 (not shown). This leads to an increase in the liquid sample 2 level in the receptacle 6. Gas arranged in the second chamber 35 flows through the holes 36 into the first chamber 35 and via the connection part 7 out of the processing device 1. The gas flow is indicated with arrows in FIG. 10.

### REFERENCE SIGNS

1 processing device
2 liquid sample
3 lid part
4 bottom part
5 hollow extension tube
6 receptacle
7 connection part
8 pressure control system
9 condenser element
10 space
11 gap
12 step portion
13 first temperature changing device
14 circumferential rim
14a rim portion
15 further connection part
17 lid part side
18 processing system
19 second temperature changing device
20 control unit
21 multiwell carrier
22 receptacle wall
23 bottom plate
24 recess
25 through hole
26 tip
27 connection portion
28 condensed liquid
29 housing
30 connection portion
31 extension
32 first wall portion
33 first chamber
34 second wall portion
35 second chamber
36 hole
37 attachment portion
E plane
L length axis

1.-38. (canceled)

39. A processing device for processing a liquid sample, the processing device comprising:
at least one condenser element for condensing of vaporized liquid of the liquid sample;
a housing that is positionable such that the condenser element is in fluid connection with a receptacle having the liquid sample, wherein the condenser element is part of the housing and/or arrangeable or arranged in a space of the housing; and
at least one temperature changing device for creating a temperature difference between the condenser element and the vaporized liquid of the liquid sample;
wherein the housing has a lid part and a bottom part and the condenser element is part of the lid part, and the bottom part comprises at least one hollow extension tube that at least partly is arrangeable in the receptacle having the liquid sample.

40. The processing device according to claim 39, wherein the condenser element is located such that condensed liquid flows back into the receptacle and/or drops on the liquid sample in the receptacle.

41. The processing device according to claim 39, wherein:
a. the processing device comprises at least one control unit for controlling the at least one temperature changing device so that the condenser element and the vaporized liquid of the liquid sample have different temperatures; and/or
b. at least one control unit controls the at least one temperature changing device such that the temperature of the condenser element is lower than the temperature of the vaporized liquid of the liquid sample.

42. The processing device according to claim 39, wherein:
a. the condenser element is part of the lid part; and/or
b. the lid part is formed in one piece; and/or
c. the condenser element is formed by deep drawing the lid part; and/or
d. the condenser element protrudes from a remaining lid part; and/or
e. the condenser element has a different thermal conductivity than the remaining lid part.

43. The processing device according to claim 39, wherein the processing device comprises a first temperature changing device for heating or cooling the lid part, wherein:
a. the first temperature changing device is placed on the lid part (3); and/or
b. the first temperature changing device covers the lid part; and/or
c. the first temperature changing device is a plate; and/or
d. the first temperature changing device comprises at least one through hole.

44. The processing device according to claim 39, wherein the condenser element corresponds to at least a part of the bottom part.

45. The processing device according to claim 39, wherein the lid part comprises a circumferential rim that surrounds a part of the bottom part.

46. The processing device according to claim 45, wherein:
a. the bottom part is directly connected to the circumferential rim; and/or
b. the extension tube extends further than the circumferential rim along a length axis of the extension tube.

47. The processing device according to claim 39, wherein the condenser element has at least one step portion that:
a. is arranged outside the hollow extension tube; and/or
b. is arranged in a plane comprising a step portion part and a space part delimited by the extension tube.

48. The processing device according to claim 39, wherein a number of extension tubes corresponds to a number of condenser elements connected to the lid part.

49. The processing device according to claim 39, wherein the lid part comprises a first wall portion, which delimits a first chamber, and a second wall portion, wherein the second wall portion and the bottom part delimit a second chamber that is fluidically connected to the first chamber.

50. The processing device according to claim 49, wherein the lid part has at least one hole for fluidically connecting the first chamber to the second chamber.

51. The processing device according to claim 50, wherein:
a. the at least one hole is arranged in the second wall portion; and/or
b. the first wall portion comprises an attachment portion for attaching the first wall portion on the second wall portion, wherein a section of the second wall portion that is delimited by the attachment portion comprises the at least one hole; and/or
c. a plane comprising the at least one hole is perpendicular to a length axis of the hollow extension tube; and/or
d. the at least one hole is arranged in a central region of the lid part; and/or
e. the at least one hole is arranged between two rim portions of the lid part; and/or
f. the one or more holes are arranged in a section of the lid part, which is arranged between two extension tubes.

52. The processing device according to claim 49, wherein:
a. the first wall portion is arranged such that gas flows from the first chamber to the second chamber when a positive pressure is applicable by a pressure control system; and/or
b. the first wall portion is arranged such that gas flows from the second chamber to the first chamber when a negative pressure is applicable by the pressure control system; and/or
c. the first wall portion is connected to the second wall portion; and/or
d. the first wall portion and the bottom part are arranged opposite to each other with respect to the second wall portion; and/or
e. the first wall portion is arranged on the second wall portion.

53. The processing device according to claim 49, wherein:
a. the second wall portion is connected to the bottom part; and/or
b. the condenser element is a part of the second wall portion; and/or
c. the connection part extends from the first wall portion.

54. A processing system with a receptacle and a processing device according to claim 39, wherein the housing is positioned such that the condenser element is in fluid connection with the receptacle.

55. The processing system according to claim 54, wherein:
a. the processing device is arranged above the receptacle; and/or
b. the circumferential rim is in contact with the receptacle.

56. The processing system according to claim 54, wherein the processing system comprises a second temperature changing device for heating or cooling the receptacle, and wherein:
a. the receptacle is arranged on the second temperature changing device; and/or
b. the receptacle covers the second temperature changing device; and/or
c. the first temperature changing device and the second temperature changing device are arranged opposite to each other with respect to the receptacle and/or the bottom part.

57. The processing system according to claim 54, wherein:
a. at least one control unit controls a first temperature changing device such that a temperature of the lid part and/or bottom part increases or decreases; and/or
b. the at least one control unit controls a second temperature changing device such that the temperature of the vaporized liquid of the liquid sample increases; and/or
c. the at least one control unit controls the first temperature changing device such that the temperature of the lid part increases and controls the second temperature changing device such that the temperature of the vaporized liquid of the liquid sample increases; and/or
d. the at least one control unit controls the first temperature changing device such that the temperature of the lid part decreases and controls the second temperature changing device such that the temperature of the vaporized liquid of the liquid sample increases.

58. The processing system according to claim 54, wherein:
a. the processing system comprises a multiwell carrier comprising a plurality of receptacles wherein an extension tube extends into each of the receptacles; and/or
b. the processing system comprises a pressure control system that is fluidically connected with the connection part of the processing device, wherein the pressure control system is configured to apply a positive pressure or a negative pressure in the space of the processing device.
